(19) United States
(12) Patent Application Publication
(10) Pub. No.: US 2007/0208392 A1
(43) Pub. Date: Sep. 6, 2007

(54) System for functional electrical stimulation

(75) Inventors: Doug Kuschner, Santa Clarita, CA (US); Dan Han, Valencia, CA (US); Morten Hansen, Valencia, CA (US); Jon Phil Mobley, Canyon Country, CA (US); Joseph H. Schulman, Santa Clarita, CA (US)

Correspondence Address: ALFRED E. MANN FOUNDATION FOR;SCIENTIFIC RESEARCH, PO BOX 905, SANTA CLARITA, CA, 91380, US

(73) Assignee: Alfred E. Mann Foundation for Scientific Research, Santa Clarita, CA (US)

(21) Appl. No.: 11/707,281

(22) Filed: Feb. 16, 2007

Related U.S. Application Data
(60) Provisional application No. 60/774,405, filed on Feb. 17, 2006.

(51) Int. Cl. A61N 1/18

(52) U.S. Cl. 607/48; 607/49

(57) ABSTRACT

An exercising system and method for the treatment and rehabilitation of the paralyzed muscles of the legs. Stimulators and sensors are implanted in several of the main muscle groups of the legs. A computerized controller uses wireless signals for communications with the implanted stimulators and sensors. The person receiving the treatment sits on an exercise machine such as a stationery bicycle, a leg press, a rowing machine or other leg exercising machine. The controller determines the position of the legs and transmits a series of commands to the stimulators to provide functional electrical stimulation (FES) to the muscles of the legs, which move the legs in a cyclical or reciprocating manner, such as that needed to pedal a bicycle. Using data provided by the implanted sensors, the controller is able to adjust the stimulation commands sent to the muscles of the legs to control the amount of force exerted by the foot, to limit user fatigue and to keep the foot in a neutral position on the pedal or footrest. Users of the system who are partially paralyzed in the legs can receive an implanted EMG sensor and the controller can synchronize the stimulation of their paralyzed leg muscles with the user's own voluntary activation of their leg muscles.

### CROSS REFERENCE TO RELATED APPLICATIONS

This application claims the benefit of U.S. Provisional Application No. 60/774,405 filed on Feb. 17, 2006 entitled: “Closed Loop Control System for Constant Speed Functional Electrical Stimulation Cycling”

### BACKGROUND OF THE INVENTION

There are a substantial number of people who have either partial or full paralysis of the legs due to spinal cord injury (SCI). In the United States, it is estimated that there are approximately 200,000 people with SCI. Each year, there are more than 10,000 new cases of SCI. SCI and its associated lower limb paralysis leads to neuromusculoskeletal disorders such as osteoporosis, disuse atrophy, spasticity, muscle and joint contractures, cardiopulmonary dysfunction, and loss of muscle endurance and metabolic function. Researchers in this area of research have proposed that the physical strain in the daily life of typical SCI individuals is insufficient to improve or maintain physical capacity. As a result, SCI individuals are at the lower end of the aerobic fitness spectrum which puts them at an increased risk for secondary diseases, such as obesity, insulin resistance, hyperglycemia, diabetes, and cardiovascular disease.

Exercise training of the legs induced by functional electric stimulation (FES) has been shown to provide many health benefits to spinal cord injured (SCI) persons including: improved cardiovascular fitness, tissue viability and glucose metabolism. Results show that the health condition measured by different criteria such as oxygen uptake, pulmonary ventilation, blood pressure and heart rate can be improved after regularly and carefully planned FES cycling. Prevention of muscle atrophy, relaxation of muscle spasms, improved circulation, and increased range of motion have been reported.

There have been three types of FES-induced exercise technology: transcutaneous stimulation with electrodes placed on the surface of the skin, percutaneous stimulation with electrodes crossing the skin, and fully implantable systems where electrodes are connected to a single implantable multi-channel stimulator (i.e. “octopus system”).

All three approaches suffer from significant limitations that confine their widespread use. The transcutaneous approach requires extensive time commitment and personal assistance to don and doff the electrodes, the repeatability of electrode placement is poor, and the pain elicited by transcutaneous stimulation in individuals with incomplete injury is a hindrance.

The percutaneous approach also requires time commitment in maintaining the site where the electrodes exit this skin. This site is prone to infection and is generally unacceptable to many subjects on principle. Although the fully implantable approach presented great promise, it failed to deliver due to its high level of invasiveness, risk of infection spreading throughout the system, and its lack of flexibility in application.

There have been two commercially available cycle ergometer systems that use FES to enable a person with SCI to exercise their legs, Ergys 2 (Therapeutic Alliances, Inc., Fairborn, Ohio) and StimMaster (Electrologic, Beavercreek, Ohio). Both systems use transcutaneous electrical stimulation (i.e. surface stimulation) to activate the hip and knee extensor muscles in a cyclic manner on a specialized stationary, recumbent cycle. While these commercial systems have proven beneficial to the health of SCI persons, the acceptance of this therapy can be increased by improving ease of use, eliminating the use of surface electrodes which cause pain in some SCI persons, and allowing these persons to use a mass-market cycle (a significantly more affordable option than the two current options).

### SUMMARY OF THE INVENTION

A system adapted for providing electrical stimulation to a plurality of muscles of a human leg, said system adapted for use with a reciprocating leg exercise device, said device having a footrest adapted to receive force applied by the foot, comprising: a plurality of sensors adapted for wireless communications and for implant in a plurality of muscles of the leg, wherein at least one of the sensors being adapted for implant in the thigh of the leg, at least another one of the sensors being adapted for implant in the shank of the leg, wherein at least one of the sensors comprises a magnetic field generator, and at least another one of the sensors comprises a magnetic field sensor; a plurality of stimulators adapted for wireless communications and for implant in said leg for stimulating a plurality of muscles of the leg, wherein at least three of the stimulators are adapted to stimulate a respective one of the: gluteus maximus, quadriceps and hamstring muscles; and a system control unit adapted for wireless communications with each of the plurality of sensors and each of the plurality of stimulators, wherein the magnetic field sensor is adapted to transmit data corresponding to the sensed magnetic field for reception by the system control unit, and wherein responsive to the data received, the system control unit transmits a stimulation command to at least one of the plurality of stimulators.

### BRIEF DESCRIPTION OF THE DRAWINGS

FIG. 1 is a simplified block diagram of a system for functional electrical stimulation (FES).

FIG. 2 is a sketch of the muscles stimulated during FES cycling in one leg.

FIG. 3 shows a diagram and a table showing the relationship between crank angle, muscles stimulated and resultant leg motion during FES of a person's legs.

FIG. 4 is a sketch of a person seated at an exercise bicycle, showing the approximate locations of implanted stimulators and sensors.

FIG. 5 is a flowchart of a method for providing exercise using FES for the legs of a person.

FIG. 6 is a flowchart of a method for determining the interior knee angle.

FIG. 7 is a flowchart of a method for maintaining a constant repetition rate for the exercise induced by FES.

FIG. 8 is a flowchart of a method for maintaining the force exerted by the foot on the footrest below a preset maximum force.

FIG. 9 is a flowchart of a method for maintaining the oxygen concentration in the blood above a minimum preset level.

FIG. 10 is a flowchart of a method for maintaining the position of the foot on the footrest in a neutral position.

FIG. 11 is a flowchart of a method for using the implanted stimulators to assist a person using FES to exercise the leg muscles

FIG. 12 is a drawing of knee angle geometry, when the sensors are equidistant from the knee.

FIG. 13 is a drawing of knee angle geometry, when the sensors are not equidistant from the knee.

### DETAILED DESCRIPTION OF THE PREFFERED EMBODIMENT

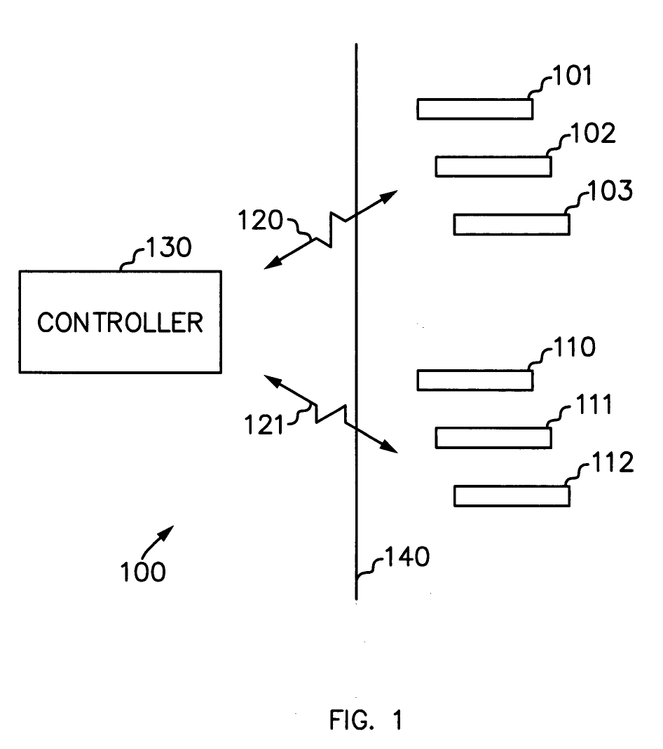

FIG. 1 is a simplified block diagram of a system 100 for functional electrical stimulation (FES). Stimulators 101, 102 and 103 and sensors 110, 111 and 112 are implanted below the skin 140 of a person. Controller 130 is a system control unit and communicates with the stimulators 101, etc. via wireless communications link 120. Controller 130 also communicates with sensors 110, etc. via wireless communications link 121. Stimulators 101, etc. and sensors 110, etc. can be battery powered and receive power from controller 130 through a coil (not shown) generating a magnetic field. Controller 130 includes a closed loop control system, which uses feedback received from a variety of sensors to control the FES during an exercise session. Implanted stimulators, implanted sensors and the wireless control of stimulators and sensors are described in U.S. Pat. Nos. 5,193,539; 5,193,540; 5,324,316; 5,405,367; 6,175,764; 6,181,965; 6,185,452; 6,185,455; 6,208,894; 6,214,032; and 6,315,721, which are incorporated herein by reference.

As used herein interchangeably, the term “leg” or the term “lower extremity” of a person includes the thigh, the knee, the shank, the ankle and the foot. As used herein, the phrase “stimulate a muscle” and similar phrases, means to stimulate a neuromuscular pathway, to stimulate a muscle's motor nerve or to stimulate a muscle's motor point, which in turn causes the muscle to contract.

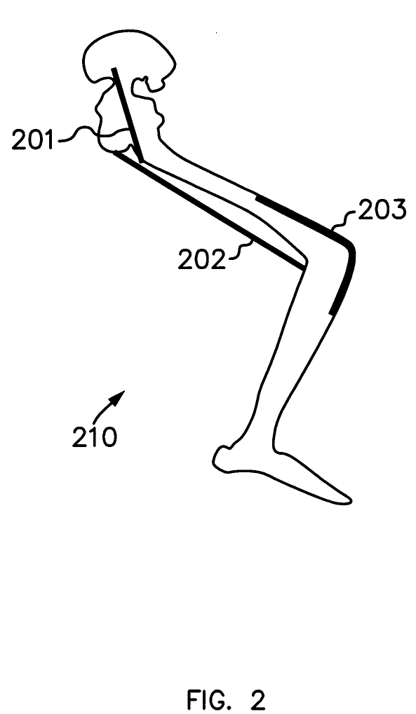

FIG. 2 is a sketch of the muscles stimulated during FES cycling in one leg, when using a stationary bicycle, such as a recumbent stationary bicycle. As has been previously discussed, there are many health benefits in exercising the paralyzed legs of a person. To move the legs of such a person in a cyclical motion, as for example, on a stationary bicycle, it has been found that only three muscles need to be stimulated in each leg 210 using FES: the gluteus maximus 201, the hamstring 202 and the quadriceps 203.

It should be noted that the diagrams and discussion of the present invention, for the purpose of simplification, are primarily with regard to one leg. With the knowledge of the motion of one leg on a bicycle, it is possible to determine the motion of the other leg, since the motions of the legs on a bicycle are 180° out of phase with each other. If the exercise machine is a leg press or a rowing machine, then the movements of the legs are in phase.

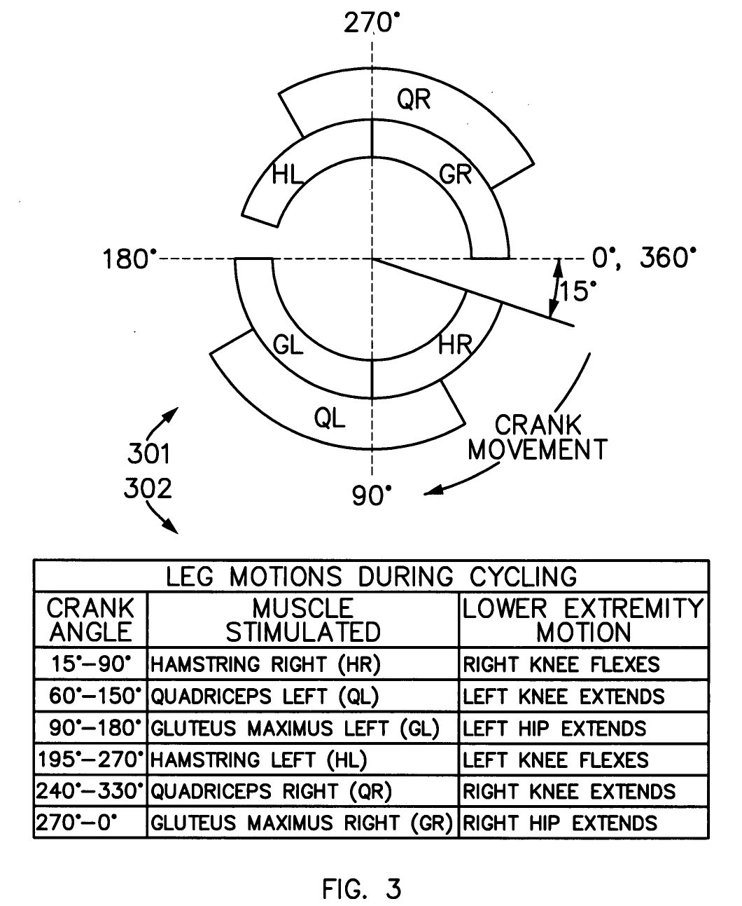

FIG. 3 shows a diagram and a table showing the relationship between crank angle of a bicycle, muscles stimulated and resultant leg motion during FES cycling of a person's legs. On a bicycle, the pedals are attached to a crank, which rotates as the pedals are depressed by the feet. The crank angle is the angle with respect to a reference line through the center of the axle of the crank. Crank angle diagram 301 in FIG. 3 shows the stimulation pattern of muscles in one cycle. In 301, the reference line is at 0°, which is to the right of the center of the circle. Table 302 shows the relationship between crank angle, the muscles stimulated and the resultant leg motion during FES of a person's legs on a bicycle. Functionally, the hamstrings are knee flexors, the quadriceps are knee extensors, and the gluteus maximus are hip extensors. In one cycle, the stimulations on the left and right legs are 180° out of phase, and the knee and hip extensors on each leg are timed to generate power through staggered phases of the movement cycle. A coordinated movement will thus be generated.

The timing of the muscle stimulation is triggered by the crank angle, and the magnitude of the stimulation can be adjusted based on crank velocity (rpm). The stimulation is in the form of electrical pulses of selectable magnitude, frequency, pulse width and commencement and termination. The stimulation can typically start 10° to 15° in advance due to the latency of muscle power in response to stimulation.

A stimulation protocol is stored by controller 130 and is transmitted to the stimulators and include such parameters as: pulse frequency, amplitude and width. The pulse frequency can be just above fusion frequency to provide smooth contractions and reduce the onset of muscle fatigue. The pulse amplitude can provide for the full range of contraction force values over the full range of pulse width parameters. The pulse width should be sufficient to offer stimulation flexibility. The stimulation protocol can be customized for each person in a fitting session, with the stimulation pulse parameters stored in a profile for that particular person.

Normally, there are three different phases of speed control needed in FES cycling: startup, cycling and cool-down. In startup phase the cycling speed is ramped up from zero to a selected value. Due to possible tendon and muscle injury caused by a sudden and intense stimulation, in the beginning of cycling the stimulation should be delivered smoothly to the person. A startup time of two minutes is typical of an FES cycling system. During startup, the stimulation intensity (the pulse width) can be increased gradually over the two minute time period. The initial intensity is zero, and the end intensity will be determined during fitting. Usually the end intensity should not be the maximum intensity that can be applied.

During the cycling phase, the controller 130 maintains a constant cycling speed, with a possible change in resistance, as a function of data received from the sensors. After the cycling phase, during the cool-down phase, the cycling speed is decreased to zero, during a time period of about two minutes.

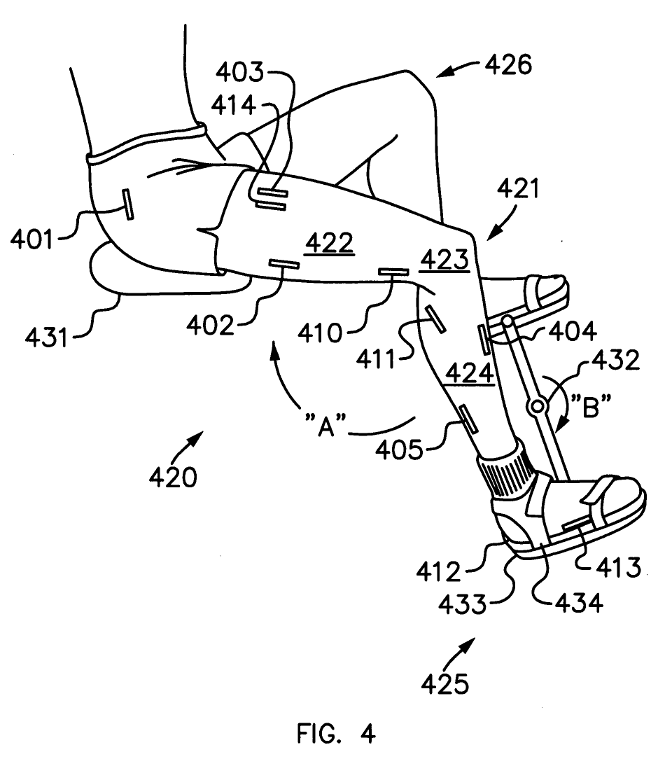

FIG. 4 is a sketch of a person 420 seated at an exercise bicycle (not shown), showing the exemplary locations of implanted stimulators and sensors in the right leg, according to an embodiment of the present invention. The implanted stimulators communicate with controller 130 via wireless communication link 120. The sensors communicate with controller 130 via communications link 121. In alternate embodiments, the implanted stimulators can function as both stimulators and sensors. A person 420 is seated on an exercise bicycle (not shown) on seat 431 with their feet on bicycle crank 432. Crank 432 can typically only move in the clockwise direction as viewed from the right side of the bicycle. Foot 425 is secured onto pedal 433 with a fastener 434, such as a boot, Velcro straps or other device. The person's right leg 421 has several stimulators and sensors implanted through the thigh 422, close to the knee 423, in the shank 424 and in or close proximity to the foot 425. Stimulators 401, 402 and 403 are implanted in the following respective muscles: gluteus maximus, hamstring and quadriceps. For the purpose of simplifying FIG. 4, the exemplary locations of stimulators and sensors in the left leg 426 are not shown.

Sensors 410 and 411 are implanted above and below the knee 423 and are used to determine the interior knee angle A as part of a magnetic goniometry system. Either one of the sensors 410 or 411 can be used as a magnetic field generator. If sensor 410 is used as a magnetic field generator, then sensor 411 can then be used as a magnetic field sensor. The strength of the sensed magnetic field is inversely proportional to the cube of the distance between the magnetic field generator and sensor, and thus a function of the interior knee angle. Data from magnetic field sensor 411 can be sent to controller 130. As the knee angle A changes, the crank speed is computed by controller 130. Given the knee angle at a point in time and knowing if the knee angle is increasing or decreasing, controller 130 then computes the crank angle B and stimulates the appropriate muscles, based on the crank angle diagram shown in FIG. 3 and the stimulation protocol for a particular person. Stimulating the leg muscles in a repeated manner through the flexion and extension of the hip, knee 423 and ankle of leg 421 will result in the cyclical motion of right leg 421 on a bicycle and provide muscle strengthening exercise. Similarly, the left leg 426 can be stimulated at the same time, but the stimulations on the left and right legs are 180° out of phase, as was discussed previously with regard to FIG. 3.

In an alternate embodiment, during a fitting session, a shaft encoder can be connected to crank 432 of the bicycle and the measured knee angle can be correlated to the measured crank angle and the results stored in a lookup table in controller 130.

In another embodiment of the present invention, pressure sensor 412 is implanted in the bottom of foot 425 and is used to measure the force exerted by foot 425 on pedal 433. Data from sensor 412 is sent to controller 130. If the force measured by sensor 412 is higher than a preset maximum, then the controller makes a change in the stimulation protocol and adjusts the stimulation commands sent to stimulators 401, 402 and 403 in order to lessen the pressure exerted by foot 425 on pedal 433 and prevent damage to foot 425. In alternate embodiments, one or more wireless pressure sensors can be installed in the bottom of fastener 434 or in pedal 433, to measure the pressure exerted by foot 425 and the resultant data sent to controller 130 can be used to lessen the force exerted by the foot 425 on pedal 433.

In an alternate embodiment of the present invention, an oxygen sensor (not shown) is used to measure the concentration of oxygen in the bloodstream of the person getting their legs exercised on a stationary bicycle using the FES system of the present invention. Data on the blood oxygen concentration level is sent to controller 130. If the oxygen concentration falls below a predetermined level, this is an indication of muscle fatigue, and controller 130 can make a change in the stimulation protocol, such as reducing the cycling speed. Various oxygen concentration sensors are known in the art and one type of oxygen sensor is described in U.S. Pat. No. 7,136,704, which is incorporated herein by reference.

In another alternate embodiment of the present invention, pressure sensor 413 is implanted close to the outside edge of foot 425 and is used to measure the lateral pressure exerted by right foot 425 on fastener 434 in order to detect a possible eversion of foot 425. In a person's use of a stationary bicycle with FES cycling, it is important to keep the legs moving in line. Being able to detect that the foot has everted or twisted to the outside is an indication that the leg is not in line as desired. Data from pressure sensor 413 is sent to controller 130. In foot eversion, the right foot 425 will twist to the outside and controller 130 can then take appropriate action to correct the position of foot 425 to a neutral position on pedal 433. Keeping foot 425 in a neutral position, i.e., pointing straight ahead with respect to the person on the bicycle, is one way of keeping the leg in a plane parallel to the sagital plane for that person. When controller 130 determines that foot eversion has taken place, then controller 130 will make a change to the stimulation protocol and adjust the stimulation of muscles 401, 402 and 403, if needed. Controller 130 will use stimulators 404 and 405 implanted respectively in the tibialis anterior and tibialis posterior in shank 424 of leg 421 to move foot 425 to a neutral position on pedal 433. In alternate embodiments of the present invention, one or more wireless pressure sensors can be installed in the side of fastener 434, to measure the lateral pressure exerted by foot 425 and the data sent to controller 130 is used to change the stimulation protocol to move foot 425 to a neutral position on pedal 433.

In an alternate embodiment of the present invention, EMG sensors (not shown) can be positioned in each of the tibialis anterior and posterior muscles. These EMG sensors would detect when the tibialis anterior and posterior are being contracted as a result of foot eversion. Data from these EMG sensors is sent to controller 130, which will determine that foot eversion has occurred and controller 130 will make a change in the stimulation protocol to move foot 425 to a neutral position on pedal 433.

In another embodiment of the present invention, electromyographic (EMG) signal sensor 414 is implanted in the quadriceps muscle, in an exemplary location for right leg 425. If person 420 is partially paralyzed and has some ability to voluntarily contract at least one of the muscles needed to cycle, such as the gluteus maximus, hamstring, quadriceps or other muscle, then sensor 414 can be positioned in a suitable location in one of those muscles. Data from sensor 414 can be processed by controller 130 to determine that a sensed EMG signal is from a voluntary contraction of a leg muscle and not from a stimulation command from controller 130. If a voluntary contraction is identified, then controller 130 can stimulate the appropriate muscles using implanted stimulators 401, 402 and 403 in a stimulation protocol to move leg 425 in a cyclical manner.

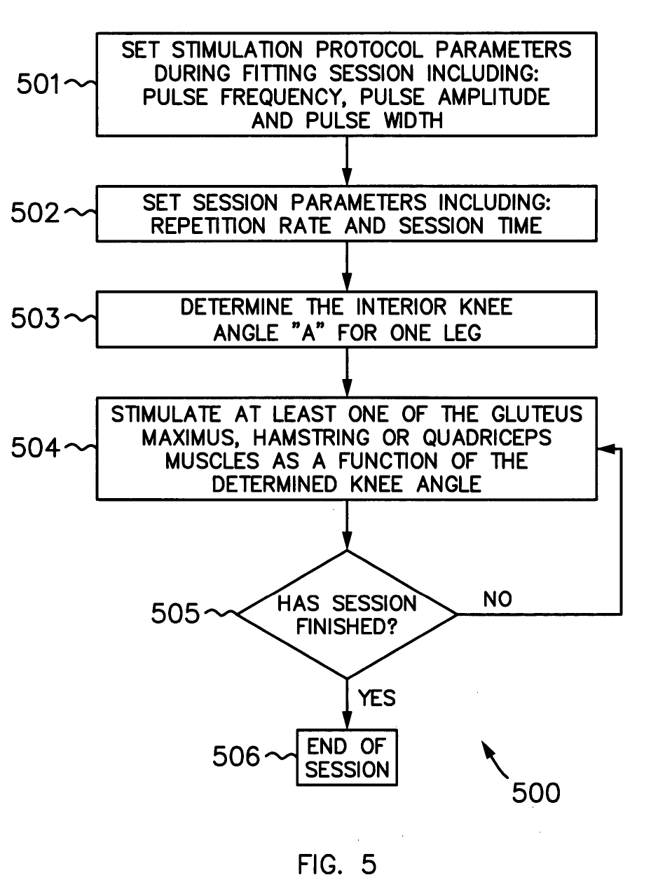

The following discussions relating to FIGS. 5-11 are with regard to methods of operation of controller 130 of the present invention. FIG. 5 is a flowchart of a method 500 for providing exercise using FES for the legs of a person. In block 501, during an initial fitting session for a person, the stimulation pulse parameters for that person are set, including: pulse frequency, pulse amplitude and pulse width. Flow proceeds from block 501 to block 502, where the session parameters are set including: repetition rate and session time. From block 502, flow, proceeds to block 503, where the interior knee angle A for one leg is determined. From block 503, flow proceeds to block 504, where controller 130 sends stimulation commands to at least one of the gluteus maximus, hamstring or quadriceps muscles as a function of the knee angle A. When controller 130 sends stimulation commands to the muscles it also considers the stimulation protocol determined in block 501. Flow proceeds to block 505 from block 504, where the elapsed time is compared to the preset stimulation session time and if the session has finished, then flow proceeds to the end of session in block 506. If the session has not ended, then flow proceeds back to block 503.

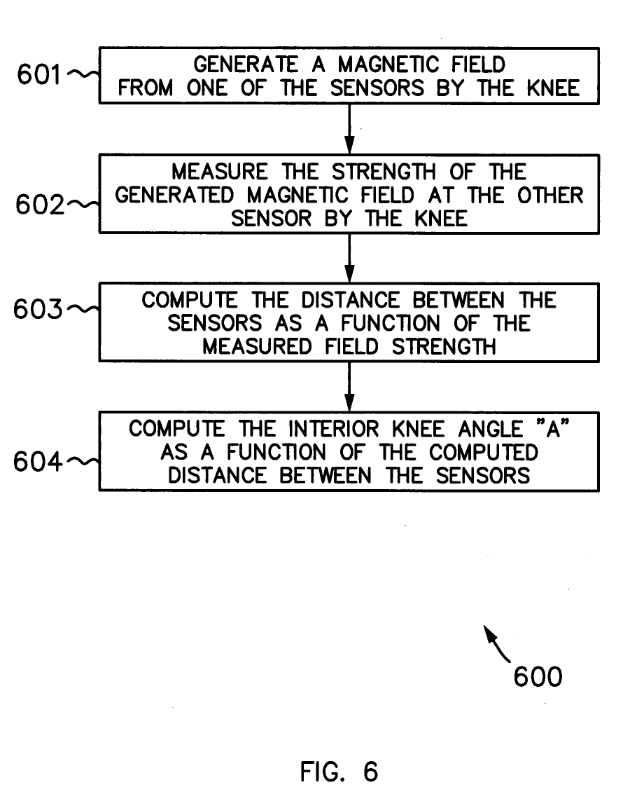

FIG. 6 is a flowchart of method 503 for determining the interior knee angle A. In block 601, Controller 130 sends a command to one of the sensors 410 or 411 next to knee 423 to generate a magnetic field. Flow proceeds to block 602, where the other sensor next to knee 423 measures the strength of the magnetic field. From block 602, flow proceeds to block 603, where controller 130 receives the data from the sensor and computes the distance between the sensors as a function of the measured magnetic field strength. After block 603, flow proceeds to block 604, where controller 130 computes the interior knee angle A as a function of the computed distance between the sensors.

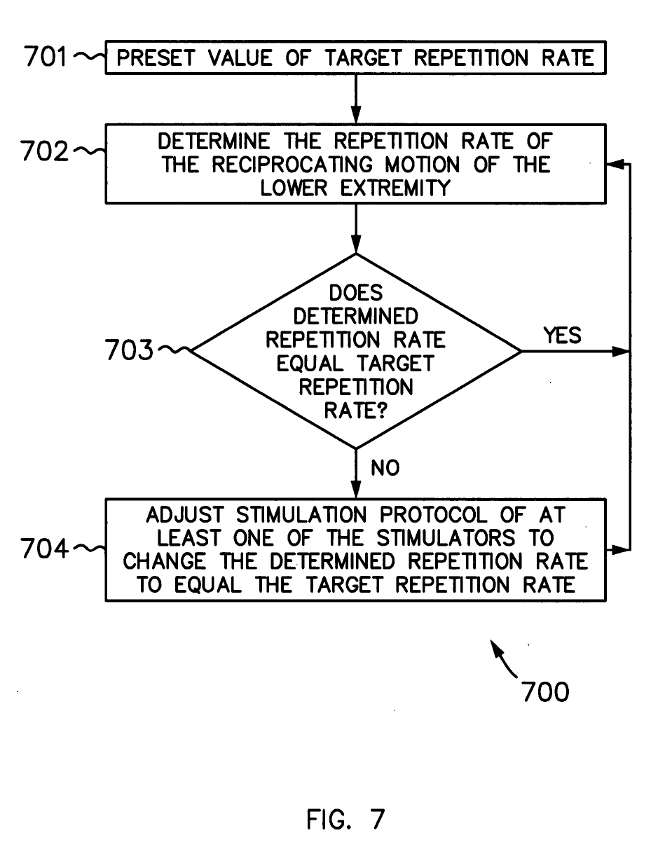

FIG. 7 is a flowchart of a method 700 for maintaining a constant repetition rate for the exercise induced by FES. In block 701, the target value of the repetition rate is preset. This step is part of the steps performed in block 502 shown in FIG. 5. In block 702, the repetition rate of the reciprocating motion of the leg is measured. From block 702, flow proceeds to block 703, where the measured repetition rate is compared to the preset target repetition rate. If the repetition rates are equal, then flow proceeds back to block 702. If the repetition rates are not equal, then flow proceeds to block 704. In block 704, the stimulation protocol of at least one of the stimulators is adjusted to change the determined repetition rate to equal the preset repetition rate, after which flow proceeds back to block 702.

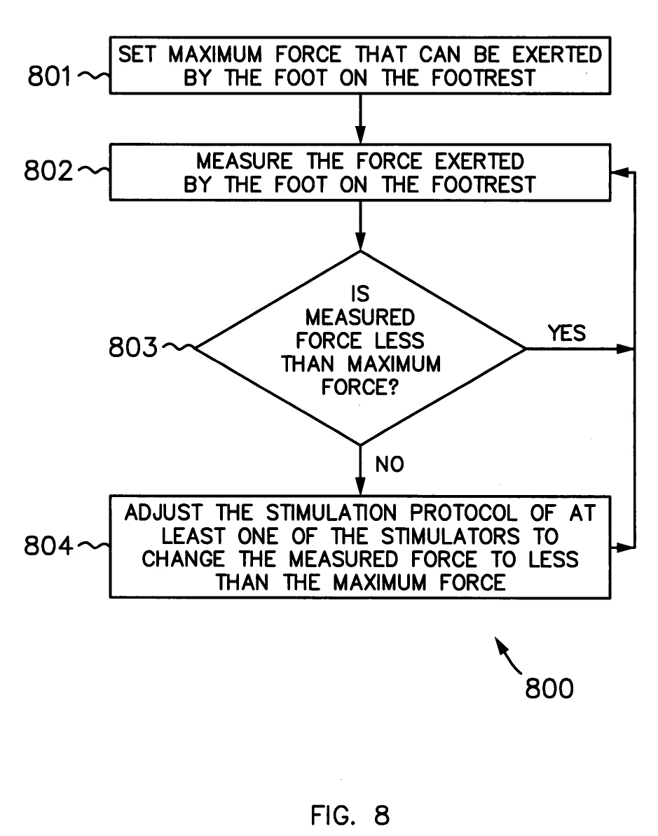

FIG. 8 is a flowchart of a method 800 for maintaining the force exerted by the foot 425 on the footrest 433 below a preset maximum force. In block 801, the maximum force that can be exerted by foot 425 on the footrest 433 is preset. This can be done during a fitting session or at the start of a session of exercise. From block 801, flow proceeds to block 802, where the force exerted by the foot 425 on footrest 433 during an exercise session is measured. After block 802, flow proceeds to block 803, where the measured force is compared to the preset maximum force. If the measured force is less than the maximum force, then flow proceeds back to block 802. If the measured force equals or exceeds the maximum force, then flow proceeds to block 804. In block 805, the stimulation protocol of at least one on the implanted stimulators is adjusted to change the measured force to less than the maximum force and then flow proceeds back to block 802.

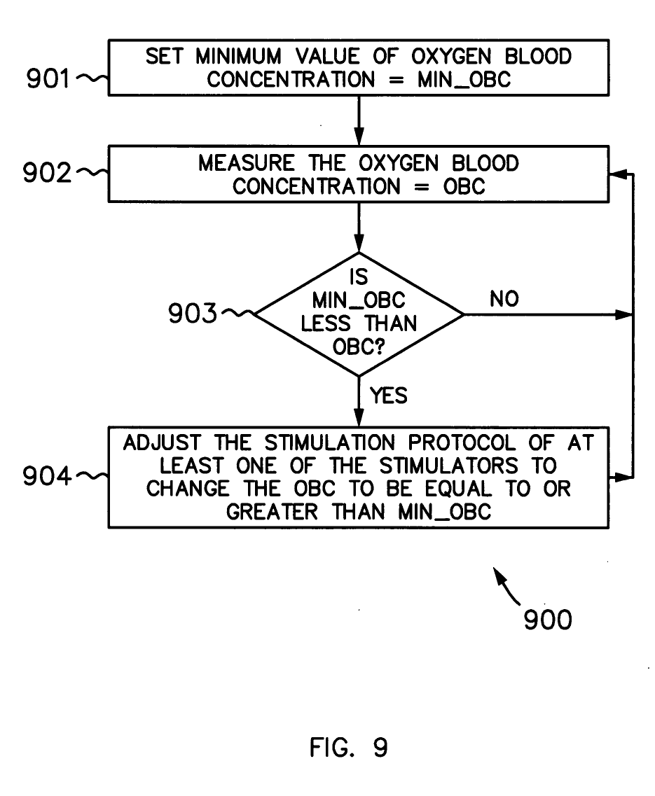

FIG. 9 is a flowchart of a method 900 for maintaining the oxygen concentration in the blood above a minimum preset level. If the blood oxygen concentration falls below a certain level, it can be an indication that the person exercising is fatigued and one or more of the parameters of the exercise session, such as the repetition rate should be decreased. In block 901, the minimum value of the concentration of oxygen in the blood is preset in a memory location, and the preset value is referred to herein as MIN_OBC, such as during a fitting session or at the start of an exercise session. From block 901 flow proceeds to block 902, where during the exercise session, the oxygen concentration in the blood is measured and the value is stored in a memory location, and the value is referred to herein as OBC. After block 902, flow proceeds to block 903, where the measured oxygen concentration (OBC) is compared with the minimum oxygen concentration (MIN_OBC). If the oxygen concentration is equal to or above the minimum oxygen concentration, then flow proceeds back to block 902. If the oxygen concentration is less than the minimum oxygen concentration, then flow proceeds to block 904, where the stimulation protocol of at least one of the implanted stimulators is adjusted to change the measured oxygen blood concentration to be greater than the minimum oxygen blood concentration. From block 904, flow returns to block 902.

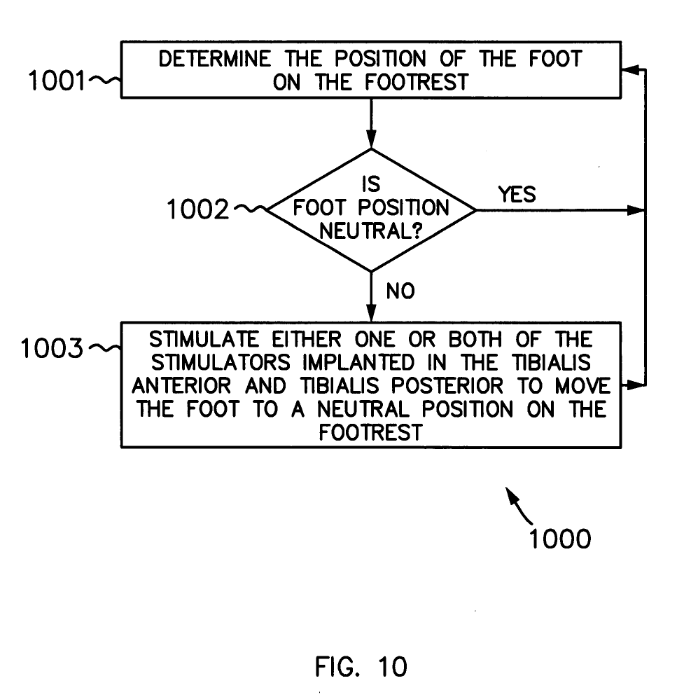

FIG. 10 is a flowchart of a method 1000 for maintaining the position of foot 425 on footrest 433 in a neutral position in order to minimize foot eversion. In block 1001, the position of foot 425 on footrest 433 is determined. This can be done using any one of a variety of sensors as was previously discussed with respect to FIG. 4. After block 1001, flow proceeds to block 1002, where the determined position of foot 425 is compared to a normal orientation of the foot on the footrest. If foot 425 is in a normal position, then flow returns to block 1001. If foot 425 is not in a normal orientation, but is in a position of eversion, then flow proceeds to block 1003, where either one or both of the tibialis anterior and posterior are stimulated to move foot 425 to a neutral orientation. After block 1003, flow returns to block 1001.

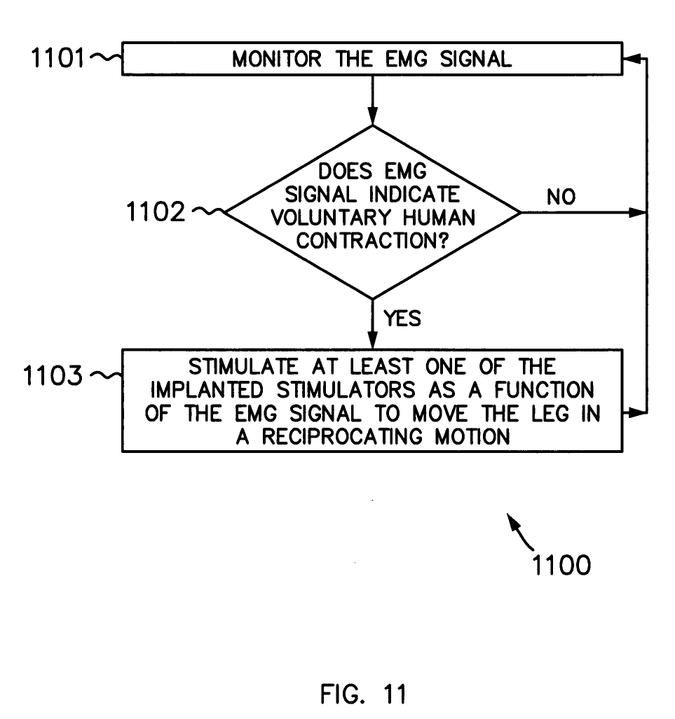

FIG. 11 is a flowchart of a method 1100 for controller 130 to use the implanted stimulators to assist a person using FES to exercise the leg muscles. In block 1101, the EMG signal from an implanted EMG sensor, such as sensor 414 for example, as discussed previously with regard to FIG. 4, is monitored. After block 1101, flow proceeds to block 1102, where the EMG signal is evaluated to determine if the muscle being monitored is contracting due to voluntary activation by the person exercising. If the EMG signal is not indicating a voluntary contraction, then flow returns to block 1101. If the EMG signal has been determined to come from a voluntary contraction by the person exercising, then flow proceeds to block 1103, where at least one of the implanted stimulators is stimulated as a function of the EMG signal to move leg 421 in a reciprocating motion. After block 1103, flow returns to block 1101.

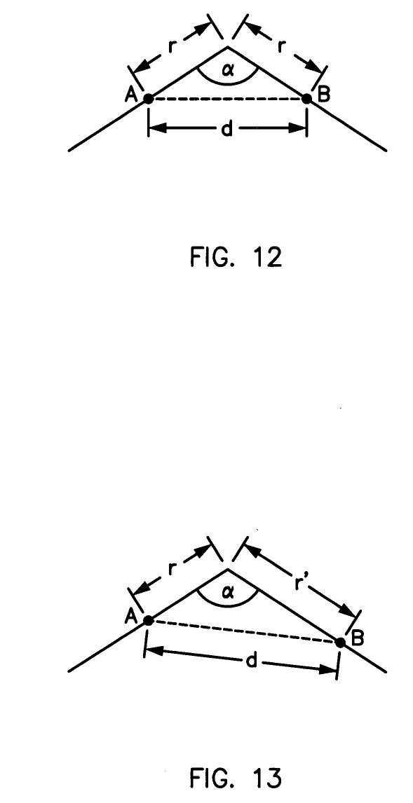

FIG. 12 is a drawing of knee angle geometry, when the sensors are equidistant from the knee, and used for an analysis of goniometry measurement accuracy. A and B are two sensors 410 and 411 implanted above and below the knee 423. The distance between each of the sensors and the knee joint=r. The distance between the two sensors=d. The interior knee angle=α. Based on the geometry:

$$d = 2 r sin ( \frac{α}{2} ) .$$

For a knee angle measurement error range of x degrees:

$$Δ d = 2 r [ sin ( \frac{α + x}{2} ) - sin ( \frac{α}{2} ) ] = 4 r sin ( \frac{x}{4} ) cos ( \frac{α + x / 2}{2} )$$

If we assume that the magnetic distance measurement system has a 1.00% error within a 10 cm range, Δd≦0.01d. The knee angle range will be determined by the following equation:

$$4 r sin ( \frac{x}{4} ) cos ( \frac{α + x / 2}{2} ) \leq 0.01 \times 2 r sin ( \frac{α}{2} ) \\ ⇒ cos ( \frac{α + x / 2}{2} ) \leq \frac{0.02}{4 sin ( \frac{x}{4} )} sin ( \frac{α}{2} )$$

The reliable knee angle range will be decided by the accuracy requirement x. For example, if x=2°, the equation above turns into:

$$cos ( \frac{α + 2}{2} ) \leq 0.573 sin ( \frac{α}{2} ) . So α < 120 ° .$$

This analysis shows that as long as the knee angle is less than 120°, then the angle measurement taken using the sensors will satisfy the 2° accuracy requirement. This can be accomplished by adjusting the distance from the seat 431 to the crank during a fitting session to limit the maximum knee angle to 120°.

FIG. 13 is a drawing of knee angle geometry, when the sensors 410 and 411 are not equidistant from the knee 423, and used for an analysis of goniometry measurement accuracy. If there is an error in implantation of the sensors 410 and 411, so that they are not equidistant from the knee joint, it is important to calculate what is the possible knee angle range to maintain a 2° accuracy in knee angle measurement. The identified items in FIG. 13 are the same as in FIG. 12, except that r′=r+Δr. In this situation the value of d is equal to:
d=\sqrt{r²+r′²−2rr′ cos α}.
For a knee angle measurement error range of x degrees:
Δd=\sqrt{r²+r′²−2rr′ cos (α+x)}−\sqrt{r²+r′²−2rr′ cos α}
To satisfy Δd≦0.01d, the inequality can be written:

$$Δ d = \sqrt{{r}^{2} + {r}^{′2} - 2 {rr}^{′} cos ( α + x )} - \sqrt{{r}^{2} + {r}^{′2} - 2 {rr}^{′} cos α} \leq 0.01 d = 0.01 \sqrt{{r}^{2} + {r}^{′2} - 2 {rr}^{′} cos α} \\ ⇒ 0.0201 ( {r}^{2} + {r}^{′2} ) \geq {rr}^{′} ( 2.0402 cos α - 2 cos ( α + x ) )$$

$$If x = 2 ° , and let y = \frac{{r}^{′}}{r} = 1 + \frac{Δ r}{r} , then : \\ 0.0201 ( 1 + {y}^{2} ) \geq y ( 2.0402 cos α - 2 cos ( α + 2 ) ) = y ( 0.0414 cos α + 0.0698 sin α ) \\ ⇒ sin ( α + 30.6 ) \leq 0.2477 \frac{1 + {y}^{2}}{y}$$

Since r+r′≦10 cm, the possible knee angle range can be calculated as below:

| r(cm) | r′(cm) | Δr(cm) | y | Knee Angle Range (degree) |
|---|---|---|---|---|
| 5 | 5 | 0 | 1 | ≦120 |
| 4.5 | 5.5 | 1 | 1.22 | ≦119 |
| 4 | 6 | 2 | 1.5 | ≦117 |
| 3.5 | 6.5 | 3 | 1.86 | ≦113 |

This analysis shows that even if the sensors are not equidistant from the knee joint, this will not cause a significant change of the knee angle range. If seat position is adjusted to ensure that the knee angle is always under 110°, an implantation location error of under 3 cm can be tolerated.

In other alternate embodiments, the exercise machine used for FES exercise can include other leg exercising machines, such as a leg press or a rowing machine, where both legs of a person go through the same flexions and extensions, at he same time. In another embodiment, it is possible to have a person with normally functioning arms and paralyzed legs row on a rowing machine using their arms and have their arm rowing motion detected by sensors. The data from the sensors are sent to controller 130, which can synchronize the stimulation of the leg muscles, to work together with the rowing motion of the arms.

Advantages of the present invention include the ability to use regular stationary exercise equipment such as a recumbent bicycle, a leg press and a rowing machine. Wireless communications to the stimulators and the sensors eliminates the problems associated with attaching electrodes to the skin of a person. The various implanted sensors provide current information which can be used to protect the person exercising using an FES system. The system can limit the amount of force exerted by the feet, to limit user fatigue and to correct the positioning of the feet if foot eversion takes place. The present invention can also assist the partially paralyzed paraplegic individual by synchronizing the stimulation of their paralyzed leg muscles with the user's own voluntary activation of their leg muscles.

While the invention herein disclosed has been described in terms of specific embodiments and applications thereof, numerous modifications and variations can be made thereto by one skilled in the art without departing from the spirit and scope of the invention as set forth in the claims.

What is claimed:

1. A system adapted for providing electrical stimulation to a plurality of muscles of a human leg, said system adapted for use with a reciprocating leg exercise device, said device having a footrest adapted to receive force applied by the foot, comprising:
a plurality of sensors adapted for wireless communications and for implant in a plurality of muscles of the leg, wherein
at least one of the sensors being adapted for implant in the thigh of the leg,
at least another one of the sensors being adapted for implant in the shank of the leg,
wherein at least one of the sensors comprises a magnetic field generator, and
at least another one of the sensors comprises a magnetic field sensor;
a plurality of stimulators adapted for wireless communications and for implant in said leg for stimulating a plurality of muscles of the leg, wherein at least three of the stimulators are adapted to stimulate a respective one of the: gluteus maximus, quadriceps and hamstring muscles;
and
a system control unit adapted for wireless communications with each of the plurality of sensors and each of the plurality of stimulators, wherein
the magnetic field sensor is adapted to transmit data corresponding to the sensed magnetic field for reception by the system control unit, and wherein
responsive to the data received, the system control unit transmits a stimulation command to at least one of the plurality of stimulators.

2. The system of claim 1, wherein the footrest comprises a pedal of a bicycle.

3. The system of claim 1, wherein the footrest comprises a footrest of a leg press.

4. The system of claim 1, wherein the footrest comprises a footrest of a rowing machine.

5. The system of claim 1, wherein the system control unit is adapted to transmit a plurality of stimulation commands to the plurality of stimulators in a predetermined sequence to cause the leg to move in flexion and extension of the hip, knee and ankle in a repetitive cycle with a predetermined number of cycles per minute.

6. The system of claim 1, wherein the system control unit is adapted to transmit a plurality of stimulation commands to the plurality of stimulators in a predetermined sequence to stimulate selected muscles, to strengthen leg muscles used in flexion and extension in a repetitive cycle with a predetermined number of cycles per minute.

7. The system of claim 1, wherein at least one of the plurality of sensors comprises a pressure sensor adapted for implant in the foot, wherein the pressure sensor is configured to sense the force applied by the foot to the footrest, and the at least one pressure sensor thereby transmits data corresponding to the sensed force for receipt by the system control unit.

8. The system of claim 7, wherein upon the receipt of said sensed force, the system control unit transmits a stimulation command to at least one of the plurality of stimulators.

9. The system of claim 8, wherein the stimulation commands cause a reduction of force applied by the foot to the footrest, when the force applied by the foot is greater than a predetermined value.

10. The system of claim 6, wherein at least one of the plurality of sensors comprises an oxygen sensor adapted to measure oxygen concentration in the bloodstream and configured to transmit data corresponding to the measured oxygen concentration to the system control unit.

11. The system of claim 10, wherein upon the receipt of data from the at least one oxygen sensor, the system control unit transmits a stimulation command to at least one of the plurality of stimulators.

12. The system of claim 11, wherein the stimulation commands are configured to increase the oxygen concentration by reducing the number of cycles per minute of flexion and extension, when the oxygen concentration is less than a predetermined value.

13. The system of claim 1, wherein:
at least one stimulator being adapted for stimulating the tibialis anterior muscle; and
at least one stimulator being adapted for stimulating the tibialis posterior muscle.

14. The system of claim 13, wherein the system control unit determines whether the foot is in a neutral orientation on the footrest and transmits a plurality of stimulation commands to the at least one stimulator in the tibialis anterior and/or to the at least one stimulator in the tibialis posterior, for moving the foot to a neutral orientation on the footrest.

15. The system of claim 1, wherein at least one of the plurality of sensors comprises an electromyographic (EMG) signal sensor of at least one of a plurality of muscles of the leg, wherein the at least one EMG sensor transmits data corresponding to the sensed EMG signal to the system control unit.

16. The system of claim 15, wherein the system control unit receives the data from the sensor adapted to sense the EMG signal and responsive to the received data determines that the sensed EMG signal was generated by human activation of a muscle corresponding to a desired motion of the leg and transmits a stimulation command to at least one of the plurality of stimulators corresponding to the human activated motion of the leg.

17. A method for electrically stimulating a plurality of muscles in a human leg, wherein at least one stimulator being implanted in each of the gluteus maximus, quadriceps and hamstring muscles, said method adapted for use with a reciprocating leg exercise device, said device having a footrest, comprising the steps of:
determining an interior knee angle of a leg with the foot of such leg on the footrest;
stimulating at least one of the gluteus maximus, quadriceps and hamstring muscles as a function of the determined interior knee angle; and
repeating the steps of determining and stimulating for moving the leg in a reciprocating motion.

18. The method of claim 17, wherein at least one stimulator being implanted in each of the thigh and shank of the leg, and wherein the step of determining the interior knee angle comprises the steps of:
generating a magnetic field from one of the sensors above or below the knee; measuring the strength of the generated magnetic field using the other one of the sensors;
computing the distance between the sensors as a function of the measured magnetic field strength; and
computing the interior knee angle as a function of the computed distance between the sensors.

19. The method of claim 17, and further comprising the steps of:
determining the repetition rate of the reciprocating motion of the leg;
comparing the determined repetition rate to a predetermined repetition rate value;
adjusting the stimulation protocol of at least one of the plurality of stimulators to change the determined repetition rate to be substantially equal to the predetermined repetition rate value; and
repeating the steps of determining, comparing and adjusting to maintain a constant repetition rate.

20. The method of claim 17, wherein at least one pressure sensor being implanted in the foot, the pressure sensor being adapted to measure the force exerted by the foot on the footrest, and further comprising the steps of:
measuring the force exerted on the footrest using the at least one pressure sensor implanted in the foot;
comparing the measured force to a predetermined maximum foot force;
adjusting the stimulation protocol of at least one of the plurality of stimulators to reduce the force exerted by the foot; and
repeating the steps of measuring, comparing and adjusting to maintain the force exerted by the foot less than the predetermined maximum foot force.

21. The method of claim 17, further comprising the steps of:
measuring oxygen concentration in the bloodstream;
comparing the measured oxygen concentration to a predetermined minimum oxygen concentration value;
adjusting the stimulation protocol of at least one of the plurality of stimulators to increase the measured oxygen concentration above the predetermined minimum oxygen concentration value; and
repeating the steps of measuring, comparing and adjusting to maintain an oxygen concentration value above the predetermined minimum oxygen concentration value.

22. The method of claim 17, wherein at least one stimulator being implanted in each of the: tibialis anterior and tibialis posterior muscles, and further comprising the steps of:
determining the position of the foot on the footrest relative to a neutral position;
stimulating either or both of the at least one stimulators implanted in the tibialis anterior and tibialis posterior muscles to move the foot to a neutral position; and
repeating the steps of determining and stimulating to maintain the foot in a neutral position on the footrest.

23. The method of claim 17, wherein at least one electromyographic (EMG) sensor being implanted in at least one muscle of the leg, and further comprising the steps of:
monitoring the sensed EMG signal;
determining if the sensed EMG signal indicates voluntary human contraction; and
stimulating at least one of the plurality of stimulators as a function of determining voluntary human contraction, to move the leg in a reciprocating motion.
